(19) United States
(12) Patent Application Publication
(10) Pub. No.: US 2026/0209146 A1
(43) Pub. Date: Jul. 23, 2026

(54) PROCESS FOR COUPLING PROPANE DEHYDROGENATION AND CO2 CAPTURE-IN SITU CONVERSION

(71) Applicant: TIANJIN UNIVERSITY, Tianjin (CN)

(72) Inventors: Jinlong Gong, Tianjin (CN); Kaige Tian, Tianjin (CN); Sai Chen, Tianjin (CN); Chunlei Pei, Tianjin (CN); Yan Li, Tianjin (CN); Jingyi Zhao, Tianjin (CN); Chenghang Liu, Tianjin (CN); Chengbo Wang, Tianjin (CN)

(21) Appl. No.: 19/254,207

(22) Filed: Jun. 30, 2025

(30) Foreign Application Priority Data
Jan. 22, 2025 (CN) 2025101027809

(51) Int. Cl. C07C 5/333; B01D 53/62; B01D 53/82; B01J 21/04; B01J 21/08; B01J 23/22; B01J 23/26; B01J 23/60; B01J 23/62; B01J 23/847; B01J 23/89; B01J 37/18; C01B 32/40

(52) U.S. Cl. CPC C07C 5/3337; B01D 53/62; B01D 53/82; B01J 21/04; B01J 21/08; B01J 23/22; B01J 23/26; B01J 23/60; B01J 23/626; B01J 23/8472; B01J 23/8966; B01J 37/18; C01B 32/40; B01D 2251/404; B01D 2257/504

(57) ABSTRACT

The present disclosure belongs to the technical field of propane production technologies and CO₂ capture and utilization, and relates to a process for coupling propane dehydrogenation and CO₂ capture-in situ conversion, including: packing a propane dehydrogenation catalyst and a CO₂ capture agent into a reactor; capturing CO₂ in a CO₂-containing atmosphere by the CO₂ capture agent; and performing a propane dehydrogenation reaction by the propane dehydrogenation catalyst and performing a CO₂ in situ conversion reaction by the CO₂ capture agent in a propane-containing atmosphere; wherein propane and hydrogen are produced by the propane dehydrogenation reaction, the hydrogen is consumed in situ by a reverse water gas reaction, and meanwhile the captured CO₂ is converted into CO in situ.

### CROSS-REFERENCE TO RELATED APPLICATION

This application claims priority from the Chinese patent application 2025101027809 filed Jan. 22, 2025, the content of which is incorporated herein in the entirety by reference.

### TECHNICAL FIELD

The present disclosure belongs to the technical field of propylene production technologies and CO₂ capture and utilization, and particularly relates to a process for coupling propane dehydrogenation and CO₂ capture-in situ conversion.

### BACKGROUND ART

Propylene is an important basic chemical raw material, which can be used to produce various high-added-value downstream products, such as acrylonitrile, acetone, propylene oxide, etc. It can be applied in multiple fields, including home appliances, textiles, building materials, plastics, automobiles, and medical materials. With the rapid growth of the downstream market, the global demand for propylene continues to increase.

There are three main traditional methods for propylene production. One is to produce propylene by petroleum cracking, which separates propylene from by-products of an ethylene device through a steam cracking process. However, the propylene productivity of this process is greatly affected by the supply of petroleum resources and the composition of cracking stocks. One is fluid catalytic cracking, which produces propylene as a by-product in the petroleum refining process. However, its productivity is relatively low and is subject to the overall fluctuation of the petroleum refining industry. And the other one is propane dehydrogenation (PDH), PDH has gradually become one of the mainstream technologies for propylene production with the rise of shale gas development. This process directly dehydrogenates propane into propylene through a catalyst, featuring high selectivity and conversion rate. The current direct dehydrogenation processes mainly include the Oleflex process of UOP Co., which mainly uses precious metal-Pt, and the Catofin process of Lummus Co., which mainly uses metal oxide-Cr₂O₃.

However, the chemical industry is a significant source of global carbon emissions, accounting for 20% or above of industrial carbon emissions. Among them, the annual carbon dioxide (CO₂) emissions are approximately 900 million tons, accounting for 7% of the global total emissions. The main sources include the combustion of fossil fuels, process emissions (such as the production of ethylene and ammonia), and waste treatment. The PDH process is a highly endothermic reaction (with a reaction enthalpy of approximately +120 KJ/mol), and it requires a high temperature (500-700° C.) to achieve a considerable propylene productivity. In industry, heat is usually supplied by the combustion of fossil fuels (such as natural gas), and the combustion process directly produces CO₂ emissions. During the PDH process, apart from the conversion of propane into propylene, cracking reactions (generating light hydrocarbons such as methane and ethylene) may also occur. If these light hydrocarbons are combusted subsequently, they will further produce CO₂. In addition, for example, in the Catofin process, a catalyst needs to be regenerated regularly to remove carbon deposits. The regeneration process is usually accomplished by combusting the carbon deposits, resulting in CO₂ emissions. If equipment used in the PDH process (such as compressors and separation devices) relies on electricity for operation and the electricity comes from fossil fuel power generation, the electricity consumption will indirectly lead to CO₂ emissions. According to industrial data, the CO₂ emissions of the PDH process are approximately 1.5-3.0 tons of CO₂ per ton of propylene.

In recent years, innovative technologies such as carbon dioxide conversion, methanol-to-propylene process, and the utilization of biomass-based raw materials have gradually attracted attention. Although these methods theoretically have a lower carbon footprint and the potential for sustainable development, their industrial application still faces challenges such as catalyst development, process stability and cost control. It can be foreseen that the PDH process will remain the main process for propylene production. However, the PDH process has problems with high energy consumption and significant environmental impact, which are the main factors restricting its green and sustainable development.

### SUMMARY

The present disclosure provides a process for coupling propane dehydrogenation and CO₂ capture-in situ conversion. A CO₂ capture agent is introduced in a propane dehydrogenation reaction process, and CO₂ capture-in situ conversion, and utilization in a chemical industry can be achieved while increasing propylene productivity through CO₂ in situ capture and conversion.

The above objective of the present disclosure is achieved by the following technical solution:

The present disclosure provides a process for coupling propane dehydrogenation and CO₂ capture-in situ conversion, including:

packing and introducing a propane dehydrogenation catalyst and a CO₂ capture agent into a reactor, to integrate a propylene-producing process with a CO₂ capture-in situ conversion process;

capturing CO₂ in a CO₂-containing atmosphere by the CO₂ capture agent; and

performing a propane dehydrogenation reaction by the propane dehydrogenation catalyst and performing a CO₂ in situ conversion reaction by the CO₂ capture agent in a propane-containing atmosphere; wherein propane and hydrogen are produced by the propane dehydrogenation reaction, the hydrogen is consumed in situ by a reverse water gas reaction, and meanwhile the captured CO₂ is converted into CO in situ.

Furthermore, the packing manner of the propane dehydrogenation catalyst and the CO₂ capture agent are selected from one of the following manners: upper and lower bed packing, particle mixed packing, and forming and packing after powder mixing; wherein the upper and lower bed packing refers to that an upper bed is packed with the propane dehydrogenation catalyst, and a lower bed is packed with the CO₂ capture agent.

Furthermore, gas captured by CO₂ is at least one selected from a group consisting of: flue gas, pure CO₂ gas, and diluted CO₂ gas.

Furthermore, the CO₂ capture agent is at least one selected from a group consisting of a Mg-based, a Ca-base, a Li-based, a Na-based, and a Sr-based.

Furthermore, the propane dehydrogenation catalyst is a reduction-free catalyst or a catalyst requiring reduction.

The reduction-free propane dehydrogenation catalyst is one of VOₓ/Al₂O₃, VOₓ/ZrO₂, Cr₂O₃/Al₂O₃, and Ga₂O₃/Al₂O₃; the VOₓ/Al₂O₃ and VOₓ/ZrO₂ are vanadium oxide-based propane dehydrogenation catalysts.

The propane dehydrogenation catalyst requiring reduction is one of PtSn/Al₂O₃, PtSn/SiO₂, PtCu/SiO₂, and PtCu/SBA-15.

Furthermore, the propane dehydrogenation catalyst is a reduction-free catalyst. The process includes the following steps:
S1: packing the reduction-free propane dehydrogenation catalyst and the CO₂ capture agent into the reactor;
S2: heating the reactor to 600-800° C. for degassing treatment in an inert atmosphere;
S3: air-cooling the reactor to 500-600° C.;
S4: maintaining the reactor at 500-600° C., and switching to a CO₂-containing atmosphere for CO₂ capture;
S5: maintaining the reactor at 500-600° C., and switching to the inert atmosphere for purging the beds of the reactor; and
S6: maintaining the reactor at 500-600° C., and switching to a propane-containing atmosphere for the propane dehydrogenation and the CO₂ in situ conversion reactions. In the process, preferably:

In the CO₂ capture process in S4, CO₂ is reaction gas, other inert gas is balance gas, a reaction pressure is normal, and a volume fraction based on CO₂ is 1-100%.

Most preferably, the volume fraction based on CO₂ is 35-45%.

In the process of the propane dehydrogenation and CO₂ in situ conversion reactions in S6, C₃H₈ is reaction gas, other inert gas is balance gas, a reaction pressure is normal, and a volume fraction based on propane is 8-30%.

Most preferably, a reaction temperature is 550° C.

Furthermore, the propane dehydrogenation catalyst is the catalyst requiring reduction. The process includes the following steps:
S1: packing the CO₂ capture agent into the reactor;
S2: heating the reactor to 600-800° C. for degassing treatment in an inert atmosphere;
S3: air-cooling the reactor to 500-600° C.;
S4: maintaining the reactor at 500-600° C., and switching to a CO₂-containing atmosphere for CO₂ capture;
S5: air-cooling the reactor to a room temperature, and taking out the CO₂ capture agent for later use;
S6: packing the propane dehydrogenation catalyst in the type of requiring reaction into the reactor;
S7: heating the reactor to 600-800° C. for degassing treatment in an inert atmosphere;
S8: air-cooling the reactor to 500-800° C., and switching an H₂-containing atmosphere for reduction treatment of the propane dehydrogenation catalyst;
S9: air-cooling the reactor to the room temperature, and taking out the reduced propane dehydrogenation catalyst for later use;
S10: packing the propane dehydrogenation catalyst obtained in S9 and the CO₂ capture agent obtained in S5 into the reactor, and heating the reactor to 500-600° C.; and
S11: maintaining the reactor at 500-600° C., and switching to a propane-containing atmosphere for the propane dehydrogenation and the CO₂ in situ conversion reactions.

Preferably, in the process:

In the CO₂ capture process in S4, CO₂ is reaction gas, other inert gas is balance gas, a reaction pressure is normal, and a volume fraction based on CO₂ is 1-100%.

Most preferably, the volume fraction based on CO₂ is 35-45%.

In the process of reduction treatment of the propane dehydrogenation catalyst in S8, H₂ is reaction gas, other inert gas is balance gas, a reaction pressure is normal, and a volume fraction based on H₂ is 10-100%.

Most preferably, a reaction temperature is 600° C.

Most preferably, the volume fraction based on H₂ is 20-40%.

In the process of the propane dehydrogenation and CO₂ in situ conversion reactions in S11: C₃H₈ is reaction gas, other inert gas is balance gas, a reaction pressure is normal, and a volume fraction based on propane is 8-30%.

Most preferably, the reaction temperature is 550° C.

Most preferably, the volume fraction based on propane is 24-25%.

The present disclosure has the following beneficial effects:

The two processes of propane dehydrogenation and CO₂ capture-in situ conversion are coupled into one process, and by consuming hydrogen produced by propane dehydrogenation in situ, the captured CO₂ is converted into CO with higher added value, as well as the purpose of increasing propylene productivity is achieved. The process in the present disclosure has the characteristics of simple and convenient improvement in existing processes, easy operation, environmental friendliness, and the like, and the CO₂ capture agent is coupled for process improvement based on existing propane dehydrogenation processes, thereby achieving the process in the present disclosure.

### BRIEF DESCRIPTION OF THE DRAWINGS

To more clearly illustrate the technical solutions in examples of the present disclosure, drawings needed to be used in the description of the examples will be simply introduced below. It is to be understood that the following drawings only illustrate some examples of the present disclosure, and thus should not be regarded as limitations to the scope of the present disclosure.

FIG. 1 is a schematic diagram of a fixed-bed reaction system constructed by the present disclosure;

FIG. 2 is a reaction flowchart of a reduction-free propane dehydrogenation catalyst according to the present disclosure;

FIG. 3 is a reaction flowchart of a propane dehydrogenation catalyst requiring reduction according to the present disclosure;

FIG. 4 is an XRD spectrum before and after a CaMnZr CO₂ capture agent captures CO₂ in Example 1 of the present disclosure;

FIG. 5 is a diagram showing reaction performance of different bed packing manners in Examples 1-5 of the present disclosure; and

FIG. 6 is a performance comparison diagram of a single propane dehydrogenation process and a process for coupling propane dehydrogenation and CO₂ capture-in situ conversion in Examples 6-11 of the present disclosure.

### DETAILED DESCRIPTION OF THE EMBODIMENTS

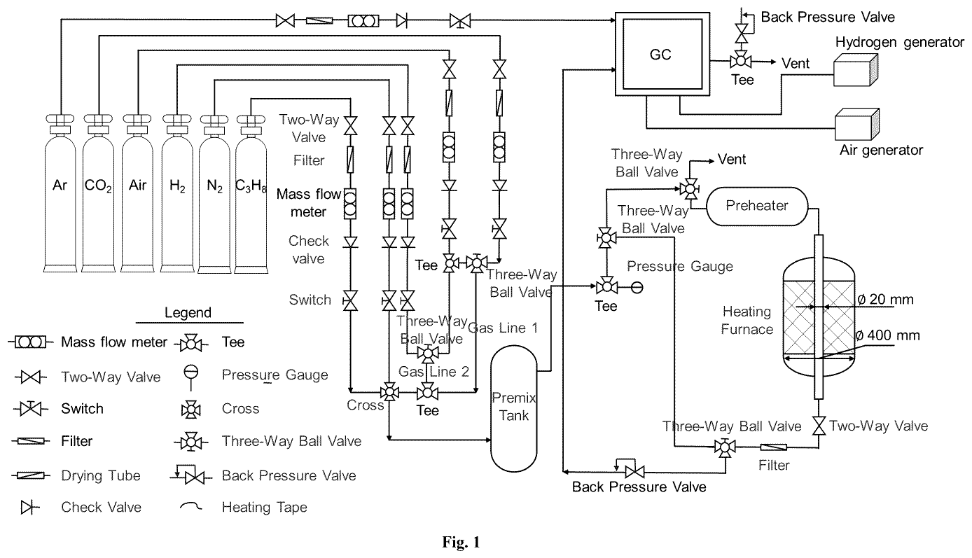

As shown in FIG. 1, a fixed-bed reaction system is autonomously designed and constructed in the present disclosure. The reaction system includes a gas cylinder, a reducing valve, steel pipelines, gas mass flow meters, a premix tank, a reaction furnace, a quartz reaction pipe, a gas chromatograph, a propane dehydrogenation catalyst, a CO₂ capture agent, and the like.

There are a total of five kinds of gases, including argon (Ar), propane (C₃H₈), hydrogen (H₂), nitrogen (N₂), carbon dioxide (CO₂), and air (Air). Wherein, Ar is used as a carrier gas for gas chromatography.

In any process, the gas firstly enters the premix tank, mixed gas enters a preheating furnace for heating, the preheated mixed gas enters the reaction heating furnace, a catalyst is packed into the quartz reaction pipe of the reaction heating furnace, and a reacted gas product enters the gas chromatograph for detection.

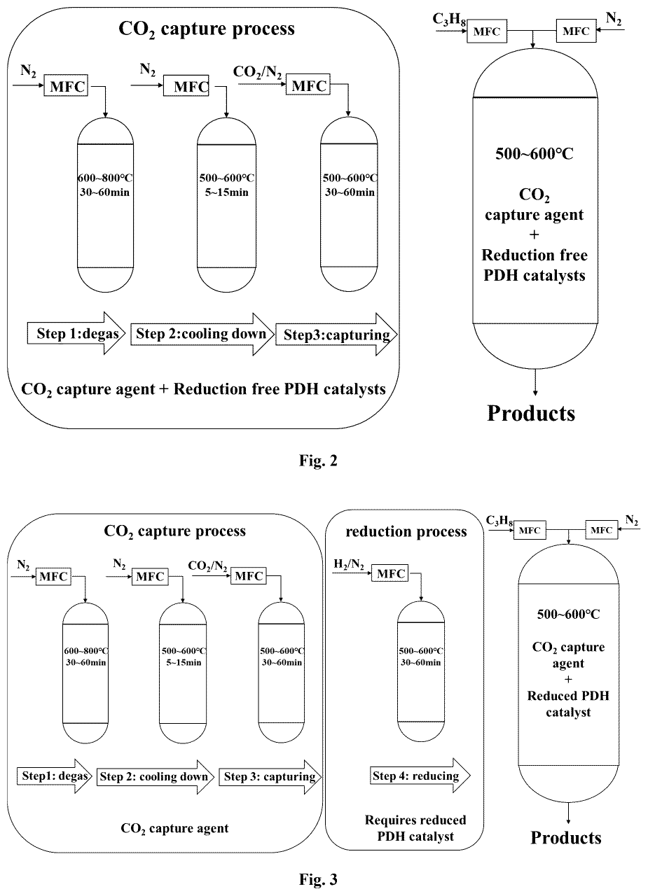

As shown in FIG. 2, for a reduction-free propane dehydrogenation catalyst, the reaction of the fixed-bed reaction system includes the following steps:
Step 1: the reduction-free propane dehydrogenation catalyst and the CO₂ capture agent are packed into a reactor after being tableted and screened.

In step 1, a tableting pressure is preferably 10 MPa, and screened propane dehydrogenation catalyst and CO₂ capture agent particles are preferably 20-40 meshes. A packing manner may be upper and lower bed packing, particle mixed packing, or powder tableted and mixed packing; wherein the upper and lower bed packing refers to that an upper bed is packed with the propane dehydrogenation catalyst, and a lower bed is packed with the CO₂ capture agent.
Step 2: an inert atmosphere is introduced from a room temperature of 20-30° C., the reactor is then heated to 600-800° C. at a programmed heating rate of 2-10° C./min, and heat preservation is performed for 30-60 minutes for degassing treatment.

In step 2, the inert atmosphere is preferably an N₂ atmosphere; the heating rate is preferably 10° C./min, and a temperature of the reactor is preferably 600° C.
Step 3: after the degassing treatment in step 2 is completed, the inert atmosphere is kept unchanged, and the heating furnace is air-cooled to 500-600° C. for purging for 10 minutes.
Step 4: after purging in step 3 is completed, the atmosphere is switched to a CO₂-containing atmosphere at a temperature of 500-600° C., and heat preservation is performed for 30-60 minutes for CO₂ capture.

In step 4, preferably, CO₂ is reaction gas, N₂ is balance gas, a reaction pressure is normal, a volume fraction based on CO₂ is 8-30%, the temperature is preferably 600° C., and heat preservation period is preferably 60 minutes.
Step 5: after step 4 is completed, the temperature of the reactor is kept at 500-600° C., and the atmosphere is switched to an inert atmosphere for purging of the beds of the reactor.
Step 6: after purging in step 5 is completed, the temperature of the reactor is kept at 500-600° C., and the inert atmosphere is switched to a propane-containing atmosphere for propane dehydrogenation and CO₂ in situ conversion reactions.

In step 6, preferably, C₃H₈ is reaction gas, N₂ is balance gas, the reaction pressure is normal, a volume fraction based on propane is 8-30%, and the temperature is preferably 550° C.
Step 7: the gas chromatograph starts to collect reaction tail gas for analysis.

As shown in FIG. 3, for a propane dehydrogenation catalyst requiring reduction, the reaction of the fixed-bed reaction system includes the following steps:
Step 1: the CO₂ capture agent is packed into the reactor after being tableted and screened.

In step 1, a tableting pressure is preferably 10 MPa, and screened CO₂ capture agent particles are preferably 20-40 meshes.
Step 2: the heating furnace is heated to 600-800° C. at a programmed heating rate of 2-10° C./min starting from a room temperature of 20-30° C., and heat preservation is performed for 30-60 minutes for degassing treatment in an inert atmosphere.

In step 2, the inert atmosphere is preferably an N₂ atmosphere; the heating rate is preferably 10° C./min, and the temperature of the reactor is preferably 600° C.
Step 3: after the degassing treatment in step 2 is completed, the inert atmosphere is kept unchanged, and the heating furnace is air-cooled to 500-600° C. for purging for 10 minutes.
Step 4: after purging in step 3 is completed, the atmosphere is switched to a CO₂-containing atmosphere at a temperature of 500-600° C., and heat preservation is performed for 30-60 minutes for CO₂ capture.

In step 4, preferably, CO₂ is reaction gas, N₂ is balance gas, a reaction pressure is normal, a volume fraction based on CO₂ is 8-30%, the temperature is preferably 600° C., and heat preservation period is preferably 60 minutes.
Step 5: after step 4 is completed, the heating furnace is air-cooled to the room temperature, and the CO₂ capture agent, which has captured CO₂, is taken out for later use.
Step 6: the propane dehydrogenation catalyst, requiring reduction, is packed into the reactor after being tableted and screened.

In step 6, a tableting pressure is preferably 10 MPa, and screened particles of the propane dehydrogenation catalyst requiring reduction are preferably 20-40 meshes.
Step 7: the heating furnace is heated to 600-800° C. at the programmed heating rate of 2-10° C./min starting from the room temperature of 20-30° C., and heat preservation is performed for 30-60 minutes for degassing treatment of the inert atmosphere.

In step 7, the inert atmosphere is preferably an N₂ atmosphere; the heating rate is preferably 10° C./min, and the temperature of the reactor is preferably 600° C.
Step 8: after the degassing treatment in step 7 is completed, the inert atmosphere is kept unchanged, and the heating furnace is air-cooled to 500-600° C. for purging for 10 minutes.
Step 9: after purging in step 8 is completed, the atmosphere is switched to an H₂-containing atmosphere at a temperature of 500-600° C., and heat preservation is performed for 30-60 minutes for reduction treatment of the propane dehydrogenation catalyst.

In step 9, preferably, H₂ is reaction gas, N₂ is balance gas, the reaction pressure is normal, a volume fraction based on H₂ is 10-100%, the temperature is preferably 600° C., and heat preservation time is preferably 60 minutes.
Step 10: after step 9 is completed, the reactor is air-cooled to room temperature, and the reduced propane dehydrogenation catalyst is taken out for later use.
Step 11: the reduced propane dehydrogenation catalyst and the CO₂ capture agent, which has captured CO₂, are mixed and then packed into the reactor.

In step 11, a packing manner may be respective packing of upper and lower beds, particle mixed packing, or powder tableted and mixed packing.
Step 12: the heating furnace is heated to 600-800° C. at the programmed heating rate of 2-10° C./min starting from the room temperature of 20-30° C.

In step 12, the inert atmosphere is preferably an N₂ atmosphere; the heating rate is preferably 10° C./min, and the temperature of the reactor is preferably 550° C.
Step 13: after the temperature in step 12 is stable, the atmosphere is switched to a propane-containing atmosphere for propane dehydrogenation and CO₂ in situ conversion reactions.

In step 13, preferably, C₃H₈ is reaction gas, N₂ is balance gas, the reaction pressure is normal, a volume fraction based on propane is 8-30%, and the temperature is preferably 550° C.
Step 14: the gas chromatograph is started to collect reaction tail gas for analysis.

The present disclosure is further illustrated below concerning the drawings of the specification and specific examples, however, the present disclosure is not limited by the examples in any form.

In all the following examples, activity of the catalyst is represented by a propane conversion rate, propylene selectivity, and productivity, which are calculated in the following formulas:
selectivity(S):

$$Sel ( \% ) = \frac{{[ {F}_{{C}_{3} {H}_{6}} ]}_{out}}{{[ {F}_{{C}_{3} {H}_{8}} ]}_{i n} - {[ {F}_{{C}_{3} {H}_{8}} ]}_{out}} \times 100$$

[F_{C}_{₃}_{H}_{₈}]ᵢₙ [symbol] [F_{C}_{₃}_{H}_{₈}]ₒᵤₜ respectively represent the propane flows at the inlet and the outlet of the reactor, with a unit of ml/min;
[F_{C}_{₃}_{H}_{₆}]ₒᵤₜ represents a propylene flow at the outlet of the reactor, with a unit of ml/min;
conversion rate (X):

$${C}_{3} {H}_{8} : Con ( \% ) = \frac{{[ {F}_{{C}_{3} {H}_{8}} ]}_{i n} - {[ {F}_{{C}_{3} {H}_{8}} ]}_{out}}{{[ {F}_{{C}_{3} {H}_{8}} ]}_{i n}} \times 100$$

Wherein, [F_{C}_{₃}_{H}_{₈}]ᵢₙ [symbol] [F_{C}_{₃}_{H}_{₈}]ₒᵤₜ represent the propane flows at the inlet and the outlet of the reactor, respectively, with a unit of ml/min;
[F_{CO}_{₂}]ᵢₙ [symbol] [F_{CO}_{₂}]ₒᵤₜ represent CO₂ flows at the inlet and the outlet of the reactor, respectively, with a unit of ml/min;
productivity:

$${C}_{3} {H}_{6} : Productivity ( {mmol}_{{C}_{3} {H}_{6}} \cdot {g}_{cat .}^{- 1} \cdot {h}^{- 1} ) = \frac{{[ {F}_{{C}_{3} {H}_{6}} ]}_{out} \times 60}{{m}_{cat .} \times 22.414}$$

[F_{C}_{₃}_{H}_{₆}]ₒᵤₜ and m_{cat} represent the propane flow at the outlet of the reactor and mass of the used catalyst, respectively, with units of ml/min and g; and
a reduction product is analyzed online by the gas chromatograph.

### Example 1

A process for coupling propane dehydrogenation and CO₂ capture-in situ conversion, including: Step (1): 0.25 g of CaMnZr particles at 20-40 meshes, as a CO₂ capture agent, are packed into the quartz pipe of the fixed-bed reactor shown in FIG. 1;
Step (2): the heating furnace is heated to 600° C. at a programmed heating rate of 10° C./min in an N₂ atmosphere from a room temperature of 25° C., and heat preservation is performed for 60 minutes for degassing treatment;
Step (3): after degassing treatment in step (2) is completed, an atmosphere is switched to a 25% CO₂/N₂ atmosphere at a temperature of 600° C., and heat preservation is performed for 60 minutes for CO₂ capture, wherein the reaction pressure is normal;
Step (4): after step (3) is completed, the heating furnace is air-cooled to room temperature, and the CO₂ capture agent, which has captured CO₂, is taken out for later use;
Step (5): 0.1 g of PtSn/SiO₂ at 20-40 meshes, as the propane dehydrogenation catalyst requiring reduction, is packed into the quartz pipe of the fixed-bed reactor;
Step (6): the heating furnace is heated to 600° C. at the programmed heating rate of 10° C./min in the N₂ atmosphere starting from the room temperature of 25° C.;
Step (7): after the temperature in step (6) is stable, an atmosphere is switched to a 10% H₂/N₂ atmosphere at a temperature of 600° C., and heat preservation is performed for 60 minutes for CO₂ capture, to perform the reduction process of the propane dehydrogenation catalyst, wherein a reaction pressure is normal;
Step (8): after step (7) is completed, the heating furnace is air-cooled to room temperature, and the reduced propane dehydrogenation catalyst is taken out for later use;
Step (9): the reduced propane dehydrogenation catalyst and the CO₂ capture agent, which has captured CO₂, are packed into the quartz pipe of the fixed-bed reactor after being mixed in a particle form;
Step (10): the heating furnace is heated to 550° C. at the programmed heating rate of 10° C./min in the N₂ atmosphere starting from the room temperature of 25° C.;
Step (11): after the temperature in step (10) is stable, the atmosphere is switched to a 40% C₃H₈/N₂ atmosphere, where the reaction pressure is normal; and
Step (12): the gas chromatograph is clicked to start to collect reaction tail gas for analysis.

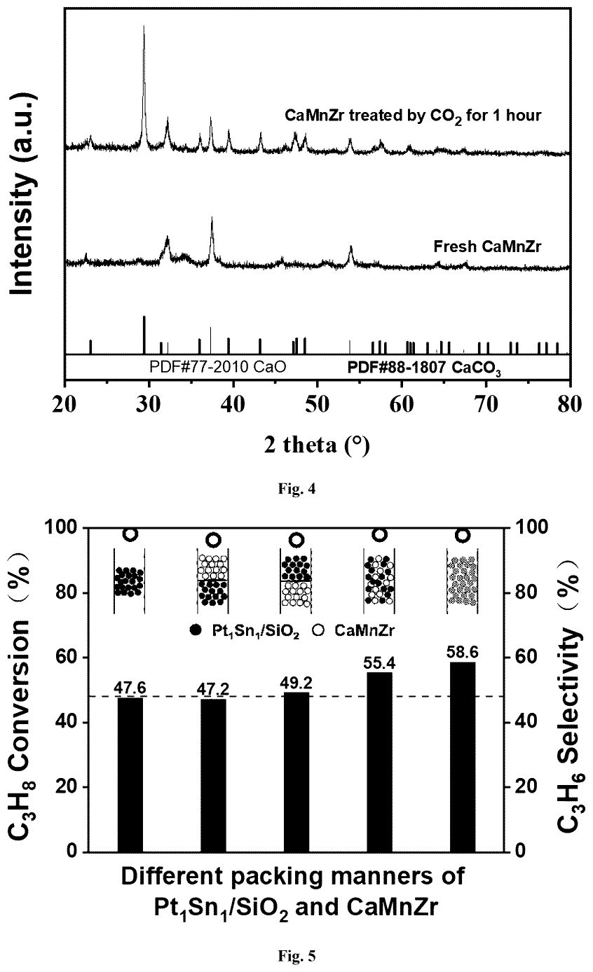

Referring to FIG. 4, comparing CaMnZr before and after CO₂ capture in Example 1, it can be shown that after CaMnZr captures CO₂, there are obvious CaCO₃ diffraction peaks in the XRD spectrum, indicating that the CO₂ capture process can well capture CO₂ and turn CaO into CaCO₃.

### Example 2

The process for coupling propane dehydrogenation and CO₂ capture-in situ conversion is performed according to the method in Example 1. Example 2 differs from Example 1 only in that in step (1), the CO₂ capture agent CaMnZr is replaced with quartz sand particles without a CO₂ capture capacity.

### Example 3

The process for coupling propane dehydrogenation and CO₂ capture-in situ conversion is performed according to the method in Example 1. Example 3 differs from Example 1 only in that in step (9), the reduced propane dehydrogenation catalyst and the CO₂ capture agent which has adsorbed CO₂ are mixed on the upper and lower beds, the CO₂ capture agent is located on the upper bed while the propane dehydrogenation catalyst is located on the lower bed, and the CO₂ capture agent and the propane dehydrogenation catalyst are packed into the quartz pipe of the fixed-bed reactor.

### Example 4

The process for coupling propane dehydrogenation and CO₂ capture-in situ conversion is performed according to the method in Example 1. Example 4 differs from Example 1 only in that in step (9), the reduced propane dehydrogenation catalyst and the CO₂ capture agent which has adsorbed CO₂ are mixed on the upper and lower beds, the propane dehydrogenation catalyst is located on the upper bed while the CO₂ capture agent is located on the lower bed, and the CO₂ capture agent and the propane dehydrogenation catalyst are packed into the quartz pipe of the fixed-bed reactor.

### Example 5

The process for coupling propane dehydrogenation and CO₂ capture-in situ conversion is performed according to the method in Example 1. Example 5 differs from Example 1 only in that in step (9), the reduced propane dehydrogenation catalyst and the CO₂ capture agent, which has adsorbed CO₂, are fully mixed after being ground into powder and tableted, and then packed into the quartz pipe of the fixed-bed reactor.

As for Examples 1-5, referring to FIG. 5, it can be shown that as for the process for coupling propane dehydrogenation and CO₂ capture-in situ conversion, compared with the addition of quartz sand without a CO₂ capture capacity, the addition of the CaMnZr capture agent with the CO₂ capture capacity can effectively increase the propane conversion rate and propylene productivity when the propane dehydrogenation catalyst is located on the upper layer while the CO₂ capture agent is located on the lower layer, when the propane dehydrogenation catalyst and CO₂ capture agent particles are mixed, and when the propane dehydrogenation catalyst and CO₂ capture agent powder is ground, tableted and mixed, and the propylene selectivity is kept unchanged; and the propane conversion rate, propylene selectivity and propylene productivity are unchanged when the CO₂ capture agent is located on the upper layer while the propane dehydrogenation catalyst is located on the lower layer. This indicates that the CO₂ capture agent does not provide additional propane activation sites in the reaction process, and pulls the propane dehydrogenation reaction to shift rightwards in a balanced manner only by consuming hydrogen produced by propane dehydrogenation in situ. This indicates that the process for coupling propane dehydrogenation and CO₂ capture-in situ conversion can achieve the purpose of increasing the propylene productivity and achieving resource utilization of CO₂.

### Example 6

The process for coupling propane dehydrogenation and CO₂ capture-in situ conversion is performed according to the method in Example 1. Example 6 differs from Example 1 only in that in step (5), 0.1 g of PtSn/SiO₂ propane dehydrogenation catalyst at 20-40 meshes is replaced with 0.02 g of PtSn/SiO₂ propane dehydrogenation catalyst at 20-40 meshes.

### Example 7

The process for coupling propane dehydrogenation and CO₂ capture-in situ conversion is performed according to the method in Example 1. Example 7 differs from Example 1 only in that in step (5), 0.1 g of PtSn/SiO₂ propane dehydrogenation catalyst at 20-40 meshes is replaced with 0.01 g of PtSnCu/SBA-15 propane dehydrogenation catalyst at 20-40 meshes.

### Example 8

The process for coupling propane dehydrogenation and CO₂ capture-in situ conversion is performed according to the method in Example 1. Example 8 differs from Example 1 only in that in step (5), 0.1 g of PtSn/SiO₂ propane dehydrogenation catalyst at 20-40 meshes is replaced with 0.02 g of PtZn/SiO₂ propane dehydrogenation catalyst at 20-40 meshes.

### Example 9

The process for coupling propane dehydrogenation and CO₂ capture-in situ conversion, including: step (1): 0.3 g of CaMnZr particles at 20-40 meshes as the CO₂ capture agent and 0.3 g of VOₓ/Al₂O₃ (vanadium oxide-based) propane dehydrogenation catalyst particles at 20-40 meshes as the reduction-free propane dehydrogenation catalyst, which are packed into the quartz pipe of the autonomously designed and constructed fixed-bed reactor in a particle mixing manner;
Step (2): the heating furnace is heated to 600° C. at a programmed heating rate of 10° C./min in an N₂ atmosphere from a room temperature of 25° C., and heat preservation is performed for 60 minutes for degassing treatment;
Step (3): after degassing treatment in step (2) is completed, an atmosphere is switched to a 25% CO₂/N₂ atmosphere at a temperature of 600° C., and heat preservation is performed for 60 minutes for CO₂ capture, wherein a reaction pressure is normal;
Step (4): after the CO₂ capture process in step (3) is completed, an atmosphere is switched to an N₂ atmosphere, and the N₂ atmosphere is kept for 10 minutes for purging treatment of the beds of the reactor;
Step (5): after purging of the beds is completed, the reactor is air-cooled to 550° C., and the atmosphere is switched to a 40% C₃H₈/N₂ atmosphere, wherein the reaction pressure is normal; and
Step (6): the gas chromatograph is started to collect reaction tail gas for analysis.

### Example 10

The process for coupling propane dehydrogenation and CO₂ capture-in situ conversion is performed according to the method in Example 9. Example 10 differs from Example 9 only in that in step (1), 0.3 g of VOₓ/Al₂O₃ (vanadium oxide-based) propane dehydrogenation catalyst at 20-40 meshes is replaced with 0.3 g of FeVO₄/SiO₂ propane dehydrogenation catalyst at 20-40 meshes.

### Example 11

The process for coupling propane dehydrogenation and CO₂ capture-in situ conversion is performed according to the method in Example 9. Example 11 differs from Example 9 only in that in step (1), 0.3 g of VOₓ/Al₂O₃ (vanadium oxide-based) propane dehydrogenation catalyst at 20-40 meshes is replaced with 0.3 g of Cr₂O₃/Al₂O₃ propane dehydrogenation catalyst at 20-40 meshes.

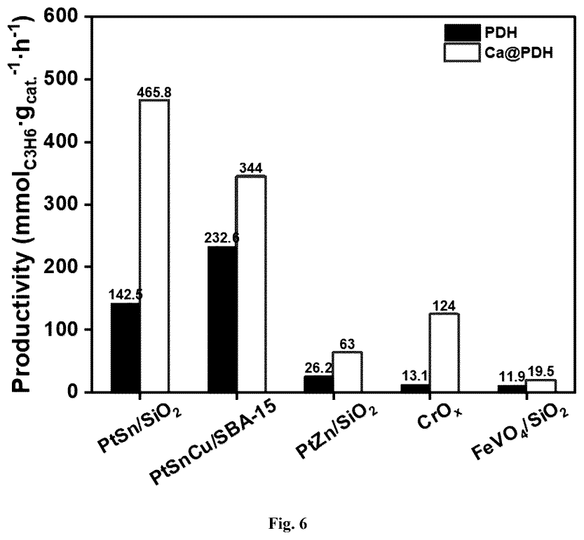

FIG. 6 shows propane productivities of different propane dehydrogenation catalysts in Examples 6-11, wherein the propylene productivity is used to represent the performance of the coupling process. It can be shown from FIG. 6 that as the reaction is performed by switching the single propane dehydrogenation process to the process for coupling propane dehydrogenation and CO₂ capture-in situ conversion, the propylene productivity is increased, indicating that the CO₂ capture agent in a coupling reaction system can effectively improve the propylene production capacity of the existing process.

In conclusion, the carbonate produced by adsorbing CO₂ by the CO₂ capture agent pulls the propane dehydrogenation reaction to shift rightwards in a balanced manner by consuming hydrogen produced by propane dehydrogenation in situ, and synthetic gas is produced by converting the captured CO₂ in situ, to achieve resource utilization of CO₂. The intensive process is achieved while increasing the propylene productivity, thereby saving energy and reducing consumption.

Although the preferred examples of the present disclosure are described concerning the drawings above, the present disclosure is not limited to the above specific implementations, and the above specific implementations are only schematic instead of restrictive. Those ordinarily skilled in the art may also make many forms of specific conversions without departing from the purpose of the present disclosure and the scope protected by the claims under inspiration of the present disclosure, and these conversions all belong to the protection scope of the present disclosure.

1. A process for coupling propane dehydrogenation and CO₂ capture-in situ conversion, comprising:
packing and introducing a propane dehydrogenation catalyst and a CO₂ capture agent into a reactor, to integrate a propylene-producing process with a CO₂ capture-in situ conversion process;
capturing CO₂ in a CO₂-containing atmosphere by the CO₂ capture agent; and
performing a propane dehydrogenation reaction by the propane dehydrogenation catalyst and performing a CO₂ in situ conversion reaction by the CO₂ capture agent in a propane-containing atmosphere;
wherein propane and hydrogen are produced by the propane dehydrogenation reaction, the hydrogen is consumed in situ by a reverse water gas reaction, and meanwhile, the captured CO₂ is converted into CO in situ.

2. The process for coupling propane dehydrogenation and CO₂ capture-in situ conversion according to claim 1, wherein packing manner of the propane dehydrogenation catalyst and the CO₂ capture agent are selected from one of the following manners: upper and lower bed packing, particle mixed packing, and forming and packing after powder mixing;
wherein the upper and lower bed packing refers to that an upper bed is packed with the propane dehydrogenation catalyst, and a lower bed is packed with the CO₂ capture agent.

3. The process for coupling propane dehydrogenation and CO₂ capture-in situ conversion according to claim 1, wherein gas captured by CO₂ is at least one selected from a group consisting of: flue gas, pure CO₂ gas and diluted CO₂ gas.

4. The process for coupling propane dehydrogenation and CO₂ capture-in situ conversion according to claim 1, wherein the CO₂ capture agent is at least one selected from a group consisting of a Mg-based, a Ca-based, a Li-based, a Na-based, and a Sr-based.

5. The process for coupling propane dehydrogenation and CO₂ capture-in situ conversion according to claim 1, wherein the propane dehydrogenation catalyst is a reduction-free catalyst or a catalyst requiring reduction.

6. The process for coupling propane dehydrogenation and CO₂ capture-in situ conversion according to claim 5, wherein the reduction-free propane dehydrogenation catalyst is one of VOₓ/Al₂O₃, VOₓ/ZrO₂, Cr₂O₃/Al₂O₃, and Ga₂O₃/Al₂O₃; the propane dehydrogenation catalyst requiring reduction is one of PtSn/Al₂O₃, PtSn/SiO₂, PtCu/SiO₂, and PtCu/SBA-15.

7. The process for coupling propane dehydrogenation and CO₂ capture-in situ conversion according to claim 5, wherein the propane dehydrogenation catalyst is the reduction-free catalyst, and the process comprises the following steps:
S1: packing the reduction-free propane dehydrogenation catalyst and the CO₂ capture agent into the reactor;
S2: heating the reactor to 600-800° C. for degassing treatment in an inert atmosphere;
S3: air-cooling the reactor to 500-600° C.;
S4: maintaining the reactor at 500-600° C., and switching to a CO₂-containing atmosphere for CO₂ capture;
S5: maintaining the reactor at 500-600° C., and switching to the inert atmosphere for purging the beds of the reactor; and
S6: maintaining the reactor at 500-600° C., and switching to a propane-containing atmosphere for the propane dehydrogenation and the CO₂ in situ conversion reactions.

8. The process for coupling propane dehydrogenation and CO₂ capture-in situ conversion according to claim 7, wherein in the CO₂ capture process in S4, CO₂ is reaction gas, other inert gas is balance gas, a reaction pressure is normal, and a volume fraction based on CO₂ is 1-100%; in the process of the propane dehydrogenation and CO₂ in situ conversion reactions in S6, C₃H₈ is reaction gas, other inert gas is balance gas, a reaction pressure is normal, and a volume fraction based on propane is 8-30%.

9. The process for coupling propane dehydrogenation and CO₂ capture-in situ conversion according to claim 5, wherein the propane dehydrogenation catalyst is the catalyst requiring reduction, the process comprises the following steps:
S1: packing the CO₂ capture agent into the reactor;
S2: heating the reactor to 600-800° C. for degassing treatment in an inert atmosphere;
S3: air-cooling the reactor to 500-600° C.;
S4: maintaining the reactor at 500-600° C., and switching to a CO₂-containing atmosphere for CO₂ capture;
S5: air-cooling the reactor to room temperature, and taking out the CO₂ capture agent for later use;
S6: packing the propane dehydrogenation catalyst in the type of requiring reaction into the reactor;
S7: heating the reactor to 600-800° C. for degassing treatment in an inert atmosphere;
S8: air-cooling the reactor to 500-800° C., and switching to an H₂-containing atmosphere for reduction treatment of the propane dehydrogenation catalyst;
S9: air-cooling the reactor to room temperature, and taking out the reduced propane dehydrogenation catalyst for later use;
S10: packing the propane dehydrogenation catalyst obtained in S9 and the CO₂ capture agent obtained in S5 into the reactor, and heating the reactor to 500-600° C.; and
S11: maintaining the reactor at 500-600° C., and switching to a propane-containing atmosphere for the propane dehydrogenation and the CO₂ in situ conversion reactions.

10. The process for coupling propane dehydrogenation and CO₂ capture-in situ conversion according to claim 9, wherein in the CO₂ capture process in S4, CO₂ is reaction gas, other inert gas is balance gas, a reaction pressure is normal, and a volume fraction based on CO₂ is 1-100%;
in the process of reduction treatment of the propane dehydrogenation catalyst in S8, H₂ is reaction gas, other inert gas is balance gas, the reaction pressure is normal, and the volume fraction based on H₂ is 10-100%;
in the process of the propane dehydrogenation and CO₂ in situ conversion reactions in S11, C₃H₈ is reaction gas, other inert gas is balance gas, the reaction pressure is normal, and the volume fraction based on propane is 8-30%.

11. The process for coupling propane dehydrogenation and CO₂ capture-in situ conversion according to claim 2, wherein the propane dehydrogenation catalyst is a reduction-free catalyst or a catalyst requiring reduction.

12. The process for coupling propane dehydrogenation and CO₂ capture-in situ conversion according to claim 3, wherein the propane dehydrogenation catalyst is a reduction-free catalyst or a catalyst requiring reduction.
